(19) United States
(12) Patent Application Publication
(10) Pub. No.: US 2010/0092574 A1
(43) Pub. Date: Apr. 15, 2010

(54) SYNERGISTIC PEROXIDE BASED BIOCIDAL COMPOSITIONS

(76) Inventor: Philip Gerdon Sweeny, Hackettstown, NJ (US)

Correspondence Address: HOFFMANN & BARON, LLP, 6900 JERICHO TURNPIKE, SYOSSET, NY, 11791, US

(21) Appl. No.: 12/565,888

(22) Filed: Sep. 24, 2009

Related U.S. Application Data
(60) Provisional application No. 61/100,326, filed on Sep. 26, 2008.

(51) Int. Cl. A01N 59/00; A01N 59/14; A01N 59/02; A01P 1/00; A01N 43/50; A01N 47/28; G01N 33/00

(52) U.S. Cl. 424/616; 424/613; 514/389; 514/588; 424/719; 436/135

(57) ABSTRACT

Disclosed is a method for controlling microbial growth in an aqueous system containing sulfite and/or bisulfite residues by addition of a peroxy compound at a pH of greater than 5. Also disclosed is a method for stabilizing an active halogen biocide in an aqueous system containing peroxide residues by addition of an N-hydrogen compound to the active halogen biocide before combining it with the peroxide containing aqueous system. Further disclosed is an optimized papermaking biocide program consisting of initially treating sulfite bleached pulp with peroxide followed by application of an N-hydrogen-stabilized active halogen compound to the paper producing white waters and an analytical method for determining peroxide concentrations in aqueous systems in the presence of sulfite and/or bisulfite.

### CROSS-REFERENCE TO RELATED APPLICATION

This application claim the benefit of priority from U.S. Provisional Patent Application No. 61/100,326 filed Sep. 26, 2008, the disclosure of which is incorporated herein by reference.

### BACKGROUND OF THE INVENTION

The invention relates to a method for controlling microbial growth in aqueous systems containing sulfite and/or bisulfite residues, such as solutions or suspensions obtained after application of sulfite-based reducing bleaches. It further relates to a method for stabilizing active halogen biocides in peroxide-containing aqueous systems.

Reducing bleaches are frequently used in paper making applications. Such bleaching processes typically employ bisulfite or bisulfite generating solutions. While enhancing paper brightness, the use of such solutions can also result in sulfite residues in the produced pulp. Sulfite residues make pulp preservation and subsequent paper machine deposit control more difficult as many major paper slimicides and preservatives such as dibromonitrilopropionamide, isothiazolinones, and, in particular, oxidizing biocides are unstable in the presence of sulfite.

### DESCRIPTION OF THE INVENTION

Surprisingly, it has been found that at optimized pH, application of oxidizing biocides to systems containing residual sulfite can not only be successful but can even provide synergistic microbial control. Specifically, it has been found that upon optimization of pH sulfite bleached pulp can be effectively, even synergistically, treated with hydrogen peroxide for enhanced bleaching and microbial control.

The rapid neutralization of hydrogen peroxide by sulfite in acidic media (pH<5) is well known and is the basis of standard hydrogen peroxide titrimetric analytical methods. It has been found that at elevated pH these normally incompatible materials can coexist for time periods sufficient for bleaching and microbial control applications.

According to the invention, microbial growth in an aqueous system containing sulfite and/or bisulfite residues is controlled by adding a peroxy compound and adjusting and maintaining a pH of greater than about 5, and in a preferred embodiment a pH of greater than about 9. Preferred embodiments of ranges of pH include a preferred range of a pH of from about 6 to a pH of about 11, and more preferably a pH of from about 7.5 to a pH of about 10. Please note that throughout this specification, quantities which are defined by numerical boundaries and ranges which have upper and lower numbers can be combined, each upper boundary with each lower boundary to define a separate range. The lower and upper boundary should each be taken as a separate element.

Preferred peroxy compounds include hydrogen peroxide, inorganic peroxy compounds such as alkali metal or alkaline earth metal perborates, percarbonates or persulfates, organic peroxy acids such as peracetic or perbenzoic acid, other organic peroxy compounds such as urea peroxide, and mixtures of the beforementioned. The term “persulfates” includes both monopersulfates (i.e., the salts of peroxymonosulfuric acid, H₂SO₅) and peroxydisulfates (i.e., the salts of peroxydisulfuric acid, H₂S₂O₈).

The efficacy of the peroxy compounds may be increased by the addition of bleach activators such as tetraacetylethylenediamine (TAED).

A particularly preferred peroxy compound is hydrogen peroxide.

The pH of the aqueous system can be controlled and/or buffered, if necessary, by addition of bases or basic salts such as alkali or alkaline earth metal hydroxides, carbonates, bicarbonates, borates, metasilicate, or mixtures thereof.

In a preferred embodiment the concentrations of sulfite and/or bisulfite and peroxy compound immediately after addition of the peroxy compound are 1 to 300 ppm each, more preferably 5 to 200 ppm and most preferred 10 to 100 ppm each.

Applications which may benefit from the sulfite/peroxide compatibilization according to the invention include pulp and papermaking, recycle paper pulping and papermaking, pulp or biomass bleaching, textile bleaching, and similar applications.

As treating aqueous systems such as pulp slurries with peroxy compounds such as hydrogen peroxide will result in a range of peroxide concentrations or residues in said aqueous systems it is important that any subsequently applied biocides be stable to the peroxide treatment or peroxide residues. It has been found that solutions containing hydrogen peroxide, such as diluted pulps for papermaking, can be successfully treated with stabilized active halogen. This additional result is unexpected as it is well known that active halogen species are neutralized by the presence of peroxides since hydrogen peroxide can act as both an oxidizing and a reducing agent.

Specifically it has been found that active halogen species with nitrogen-bound halogen are surprisingly stable in the presence of peroxides. According to the invention, an active halogen biocide in an aqueous system containing peroxides or peroxide residues is stabilized by adding an N-hydrogen compound to the active halogen biocide before combining the biocide with the peroxide containing aqueous system. Here and herein below, an N-hydrogen compound is an organic or inorganic compound having at least one hydrogen atom directly bound to a nitrogen atom.

Application areas where both peroxides and active halogen have found utility are those most suited to this novel approach.

Active halogen biocides are biocides containing halogen, in particular chlorine or bromine, in the oxidation state 0 or +1, such as elemental chlorine or bromine and hypochlorite or hypobromite.

In a preferred embodiment the concentration of active halogen (as Cl₂) stabilized by an N-hydrogen compound is 0.1 to 20 ppm. Here and herein below, the expression “as Cl₂” denotes the concentration of elemental chlorine that is stoichiometrically equivalent to the concentration of active halogen in a given system.

Preferred N-hydrogen compounds are selected from the group consisting of ammonia, ammonium salts, such as ammonium sulfate and ammonium bromide, other nitrogen compounds containing no carbon-hydrogen bonds, such as urea, biuret, isocyanuric acid, and sulfamic acid, organic N-hydrogen compounds such as p-toluenesulfonamide, 5,5-dialkylhydantoins, methanesulfonamide, barbituric acid, 5-methyluracil, imidazoline, pyrrolidone, morpholine, acetanilide, acetamide, N-ethylacetamide, phthalimide, benzamide, succinimide, N-methylolurea, N-methylurea, acetylurea, methyl allophanate, methyl carbamate, phthalohydrazide, pyrrole, indole, formamide, N-methylformamide, dicyanodiamide, ethyl carbamate, 1,3-dimethylbiuret, methylphenylbiuret, 4,4-dimethyl-2-oxazolidinone, 6-methyluracil, 2-imidazolidinone, ethyleneurea, 2-pyrimidone, azetidin-2-one, 2-pyrrolidone, caprolactam, phenylsulfinimide, phenylsulfinimidylamide, diaryl- or dialkylsulfinimides, isothiazoline-1,1-dioxide, hydantoin, glycine, piperidine, piperazine, ethanolamine, glycinamide, creatine, and glycoluril.

More preferably the N-hydrogen compound is 5,5-dimethylhydantoin, urea, ammonia, or an ammonium salt.

The peroxide or peroxide residue in the aqueous system is preferably hydrogen peroxide, an alkali metal or alkaline earth metal percarbonate, perborate, or persulfate, an organic peroxy acid, or a mixture of two or more of the beforementioned, hydrogen peroxide being most preferred.

Preferred applications of either finding, namely the synergistic performance of peroxide treated sulfite pulps and the stabilization of active halogen against degradation by peroxides or peroxide residues, are in pulp and paper processing, recycle pulping and papermaking, deinking, pulp bleaching, biomass bleaching, textile bleaching or clay slurry bleaching. Preferred aqueous systems are pulp and papermaking slurries and liquors, recycle pulp slurries, pulp thick stock, deinking pulp slurries, pulp or biomass bleaching slurries and liquids, textile bleaching liquids and clay slurries.

Other preferred applications are in water treatment such as waste water, papermaking liquors and waters, pool and spa waters, industrial cooling waters, waters exposed to reverse osmosis filters or ion exchange resins, and aqueous systems in oil field applications, including fractionation tanks and down hole applications, or hard surface disinfection.

Still other preferred applications are in aqueous systems found in food and crop protection applications, including fruit and vegetable washes, meat and poultry processing, beverage processing, fish farming, and aquaculture.

Combining the two findings, namely the synergistic performance of peroxide treated sulfite pulps and the stabilization of active halogen against degradation by hydrogen peroxide or peroxide residues leads to the definition of a highly cost-effective microbial control program for papermaking. This program comprises pulp bleaching with sulfite followed by peroxide treatment and subsequent conversion of the pulp into paper in the presence of an active halogen biocide with nitrogen-bound halogen.

In a preferred embodiment, the aqueous system containing peroxides is obtained by the addition of a composition comprising at least one peroxy compound to said aqueous system at a pH greater than about 5.

Preferred applications of the combined methods are those wherein the aqueous system is selected from the group consisting of pulp and papermaking slurries, recycle pulp slurries, pulp thick stock, deinking pulp slurries, pulp or biomass bleaching slurries and liquids, textile bleaching solutions, and clay slurries.

According to the invention, optimized cost performance can be achieved through the co-application of sulfite and peroxy compounds, optionally in combination with activators such as tetraacetylethylenediamine, co-application of peroxy compounds with active halogens, or co-application of sulfite and peroxy compounds followed by co-application or generation of peroxy compounds with active halogens. Such co-applications have been prohibited to date by the rapid mutual neutralization of these species. The current invention demonstrates methodologies for utilizing these classes of compounds cooperatively and even synergistically.

Another object of the invention is an analytical method for determining peroxide concentrations in aqueous systems in the presence of sulfite and/or bisulfite. The method comprises the steps of:
(i) adding a defined excess of an N-hydrogen-stabilized active chlorine compound to immediately destroy the sulfite and/or bisulfite while leaving an amount of unreacted N-hydrogen stabilized active chlorine compound,
(ii) measuring the amount of unreacted N-hydrogen-stabilized active chlorine compound to determine the sulfite and/or bisulfite concentration, and
(iii) determining the peroxide concentration.

The amount of unreacted N-hydrogen-stabilized active chlorine compound in step (ii) may be measured by any method known in the art, in particular by the well-known DPD method according to ISO 7393-2. The sulfite and/or bisulfite concentration corresponds to the difference of the amount of N-hydrogen-stabilized active chlorine compound added in step (i) and the amount of unreacted N-hydrogen-stabilized active chlorine compound measured in step (ii).

The determination of the peroxide concentration in step (iii) can be achieved by one of the methods known in the art, for example by titration with thiosulfate using potassium iodide as indicator.

A preferred N-hydrogen-stabilized active chlorine compound to be used in the above analytical method is 1-chloro-5,5-dimethylhydantoin (MCDMH).

The following non-limiting examples are intended to illustrate the invention in more detail.

### EXAMPLES

The expression “1 g cfu/mL” denotes the common (decadic) logarithm of the number of colony-forming units per milliliter or, in connection with the term “reduction”, the common logarithm of the quotient of the number of colony-forming units per milliliter before treatment and the number of colony-forming units after treatment. Unless otherwise indicated all concentrations in percent or ppm are expressed on a weight basis.

### Example 1

Aqueous solutions containing sodium sulfite and hydrogen peroxide were mixed at 21° C. to obtain a solution having a sulfite content (as SO₃²⁻) of 40 ppm, a hydrogen peroxide content of 20.0 ppm and a pH of 6.7. The temperature of the solution was maintained at 21° C. and the residual sulfite and peroxide content was determined at 15, 30 and 60 minutes after mixing. The procedure consisted of adding a known amount of 1-chloro-5,5-dimethylhydantoin (MCDMH) to the samples in excess of the estimated residual sulfite content. The remaining MCDMH concentration was then measured by standard DPD total halogen methodologies. As sulfite rapidly neutralizes MCDMH at all pHs the sulfite concentration is the concentration of MCDMH added less the concentration of MCDMH measured, see Equation 1 below. This procedure is valid in the presence of H₂O₂ as H₂O₂ does not react with MCMDH and does not interfere with the total active halogen method as it is run at approximately neutral pH.

[Sulfite]=[MCDMH_{added}]−[MCDMH_{measured}] (1)

The H₂O₂ concentration was determined by recording the concentration of H₂O₂ measured using acidic thiosulfate titration with KI indicator (HACH HYP-1 hydrogen peroxide test kit—Hach Co., Loveland, Colo.). Since this titration is run at acidic pH, this method yields the concentration of H₂O₂ in excess of the sulfite concentration contained in the sample. As the sulfite concentration is known from the MCMDH analysis and Equation 1, the H₂O₂ concentration can be calculated using the following Equation 2:

[H₂O₂]=[H₂O_{2 measured}]+[Sulfite_{calculated}] (2)

The estimated error in the methodology is ±1 ppm

The results are shown in Table 1 which reveals that a significant residual concentration of both materials is observed even after a period of 30 minutes.

TABLE 1

| | Sulfite (as SO₃²⁻) | Sulfite (as H₂O₂) | |
|---|---|---|---|
| Time [min] | [ppm] | [ppm] | H₂O₂ [ppm] |
| 0 | 40.0 | 17.0 | 20.0 |
| 15 | 7.2 | 3.1 | 6.1 |
| 30 | 5.4 | 2.3 | 5.1 |
| 60 | 0.0 | 0.0 | 2.4 |

### Example 2

The procedure of Example 1 was repeated with the difference that the pH of the mixed solution was 9.0 and the residual concentrations were determined 5, 15, 30, 60, 120 and 1080 minutes after mixing. The results are shown in Table 2 which demonstrates that the co-stability of hydrogen peroxide and sulfite is even further enhanced at pH 9.0 where a significant residual concentration of both peroxide and sulfite was observed even after a period of 2 h.

TABLE 2

| | Sulfite (as SO₃²⁻) | Sulfite (as H₂O₂) | |
|---|---|---|---|
| Time [min] | [ppm] | [ppm] | H₂O₂ [ppm] |
| 0 | 40.0 | 17.0 | 20.0 |
| 5 | 18.7 | 7.9 | 16.9 |
| 15 | 16.0 | 6.8 | 14.8 |
| 30 | 15.7 | 6.7 | 14.7 |
| 60 | 14.6 | 6.2 | 15.2 |
| 120 | 12.3 | 5.2 | 13.2 |
| 1080 | 0.3 | 0.1 | 8.1 |

### Example 3

Synergistic biocidal performance upon co-application of sulfite with hydrogen peroxide at elevated pH was investigated. The sulfite and peroxide concentrations indicated in Table 3 below were added to an aqueous solution made from: (a) deionized water, (b) NaHCO₃ to achieve a carbonate buffer concentration of 200 ppm (as CaCO₃ total alkalinity), (c) sulfite bleached pulp slurry to achieve a final consistency of 0.05%, carrying an associated minimal concentration of residual sulfite of 6 ppm, and (d) NaOH to achieve a pH of 9.0.

The microbial population was that provided by preparing the pulp slurry 24-48 h prior to testing and storing at room temperature, thus allowing microbial growth to a high test level. The untreated control populations were 1 g cfu/mL=5.9 for the 3 h contact test and 1 g cfu/mL=6.5 for the 24 h contact test. Populations reported are total aerobic counts using tryptone soy agar plating. The test results are shown in Table 3.

TABLE 3

| | | | | | Excess | | |
|---|---|---|---|---|---|---|---|
| | Sulfite | Sulfite | Peroxide | Peroxide: | Peroxide | | |
| | (as SO₃²⁻) | (as H₂O₂) | (as H₂O₂) | Sulfite | (as H₂O₂) | 3 h Reduction | 24 h Reduction |
| Test No. | [ppm] | [ppm] | [ppm] | Molar Ratio | [ppm] | [lg cfu/mL] | [lg cfu/mL] |
| 1 | 0 | 0 | 0 | — | | 0.00 | 0.00 |
| 2 | 32 | 14 | 0 | — | | −0.40 | 0.11 |
| 3 | 128 | 55 | 0 | — | | −0.50 | −0.45 |
| 4 | 0 | 0 | 40 | — | 40 | 1.2 | 5.5 |
| 5 | 0 | 0 | 160 | — | 160 | 3.5 | 5.5 |
| 6 | 32 | 14 | 40 | 2.9 | 26 | 0.73 | 0.05 |
| 7 | 32 | 14 | 160 | 11 | 146 | 3.8 | 5.5 |
| 8 | 128 | 55 | 40 | 0.7 | −15 | −0.01 | −0.08 |
| 9 | 128 | 55 | 160 | 2.9 | 105 | 5.0 | 5.5 |

It appears that the presence of sulfite alone had no significant effect on bacterial populations at 32-128 ppm sulfite concentrations. Hydrogen peroxide in contrast demonstrated a slowly developing level of biocidal efficacy yielding 1 g cfu/mL reductions of 1.2-3.5 in 3 h and 5.5 in 24 h. Surprisingly, at 3 h contact some mixed sulfite/hydrogen peroxide systems (Test Nos. 7 and 9) provided greater efficacy than hydrogen peroxide alone (Test No. 5).

The observed level of performance demonstrates a clear synergistic effect of sulfite and peroxide at elevated peroxide concentrations. As sulfite alone has no biocidal efficacy, the observed enhanced efficacy of hydrogen peroxide in the presence of sulfite is a result of synergy. This result can be quantified using the method of Kull et al. (F. C. Kull, P. C. Elisman, H. D. Sylwestrowicz and P. K. Mayer, Appl. Microbiol., 1961, 9, 538) which specifies that synergy is demonstrated when a synergy index (SI) according to Equation 3 of less than 1.0 is observed.

SI=(level of A)/(efficacious level of A)+(level of B)/(efficacious level of B) (3)

Setting A as the sulfite concentration and B as the peroxide concentration the following result is achieved: As sulfite is essentially non-biocidal the denominator of the first term becomes infinite and the value of the first term zero. If we set the efficacy level as the level that produces a 1 g cfu/mL reduction of 3.5 in 3 h, the denominator of the second term becomes 160 ppm (according to Test No. 5, Table 3). Synergy indices of less than 1.0 are thus achieved for Test Nos. 7 and 9 at 3 h contact according to Equation 4 below, as these tests produced greater than the target 1 g cfu/mL reduction of 3.5 associated with 160 ppm of hydrogen peroxide alone.

SI=0+(<160)/160=(<1.0) (4)

### Example 4

Synergy upon co-application of sulfite with hydrogen peroxide at higher concentrations of sulfite and hydrogen peroxide was investigated. The conditions were the same as in Example 3. The microbial population of the untreated control was 1 g cfu/mL=6.26 for the 3 h contact test and 1 g cfu/mL=6.18 for the 24 h contact test. The results are shown in Table 4.

TABLE 4

| | | | | | Excess | | |
|---|---|---|---|---|---|---|---|
| | Sulfite | Sulfite | Peroxide | Peroxide: | Peroxide | | |
| | (as SO₃²⁻) | (as H₂O₂) | (as H₂O₂) | Sulfite | (as H₂O₂) | 3 h Reduction | 24 h Reduction |
| Test No. | [ppm] | [ppm] | [ppm] | Molar Ratio | [ppm] | [lg cfu/mL] | [lg cfu/mL] |
| 1 | 0 | 0 | 0 | — | 0 | 0 | 0 |
| 2 | 128 | 55 | 0 | — | 0 | 0.32 | 0.11 |
| 3 | 512 | 218 | 0 | — | 0 | 0.08 | −0.45 |
| 4 | 0 | 0 | 120 | — | 120 | 4.0 | 4.5 |
| 5 | 0 | 0 | 150 | — | 150 | 4.2 | 3.7 |
| 6 | 0 | 0 | 160 | — | 160 | 3.3 | 5.0 |
| 7 | 128 | 55 | 120 | 2.2 | 65 | 2.3 | 3.9 |
| 8 | 32 | 14 | 150 | 11 | 136 | 4.5 | 4.3 |
| 9 | 32 | 14 | 120 | 8.6 | 106 | 4.6 | 3.6 |
| 10 | 128 | 55 | 160 | 2.9 | 105 | 3.5 | 5.3 |
| 11 | 32 | 14 | 160 | 11 | 128 | 4.5 | 5.3 |

As shown in Table 4, the application of sulfite at concentrations of 128-512 ppm has no significant effect on the microbial populations. Hydrogen peroxide at concentrations of 120-160 ppm in contrast demonstrates a slowly developing level of biocidal efficacy yielding 1 g cfu/mL reductions of 3.3-4.0 in 3 h and 3.7-5.5 in 24 h. Again surprisingly some mixed sulfite/hydrogen peroxide systems provided greater efficacy than hydrogen peroxide alone. The observed level of performance demonstrates a clear synergistic effect of sulfite and peroxide at elevated peroxide concentrations. As sulfite by itself exhibits no biocidal efficacy the observation of enhanced efficacy of hydrogen peroxide in the presence of sulfite is result of synergy. A completely rigorous demonstration of synergy is possible for Test No. 9. If the desired effect is set as 1 g cfu/mL reduction of 4.2 we can see that >512 ppm sulfite would be required to achieve this. The amount of hydrogen peroxide alone that it would take to achieve this is 150 ppm or greater. This produces Equation 5:

SI=32/(>512)+(<120)/150=(<0.063)+(<0.8)=(<0.86) (5)

### Example 5

The bactericidal efficacy of solutions containing sulfite and hydrogen peroxide was further investigated in the absence of pulp. Efficacy was measured against Pseudomonas aeruginosa grown in nutrient in the presence of 83 and 830 ppm sulfite. The sulfite-containing P. aeruginosa inoculum was then diluted 1:99 with Butterfield's buffer at pH 7.0. The sulfite concentrations in Table 5 below are the those in the final dilution. The dilutions were then contacted with 50 ppm hydrogen peroxide for 3 h at 37° C. The untreated control populations (Test 1) were 1 g cfu/mL=6.0. The test results are shown in Table 5.

TABLE 5

| | | | | | Excess | |
|---|---|---|---|---|---|---|
| | Sulfite | Sulfite | Peroxide | Peroxide: | Peroxide | |
| | (as SO₃²⁻) | (as H₂O₂) | (as H₂O₂) | Sulfite | (as H₂O₂) | 3 h Reduction |
| Test No. | [ppm] | [ppm] | [ppm] | Molar Ratio | [ppm] | [lg cfu/mL] |
| 1 | 0 | 0 | 0 | — | | 0.0 |
| 2 | 8.3 | 4 | 0 | — | | 0.1 |
| 3 | 0.83 | 0.4 | 0 | — | | 0.0 |
| 4 | 0 | 0 | 50 | — | 50 | 0.9 |
| 5 | 8.3 | 4 | 50 | — | 46 | 1.5 |
| 6 | 0.83 | 0.4 | 50 | — | 50 | 0.8 |

As shown in Table 5, the biocidal efficacy of hydrogen peroxide against P. aeruginosa grown up in 830 ppm sulfite diluted to 8.3 ppm during application (1 g cfu/mL reduction of 1.5) was surprisingly greater than that observed against P. aeruginosa grown up in the absence of sulfite (1 g cfu/mL reduction of 0.9). Thus, the surprising enhancement of hydrogen peroxide bactericidal efficacy by the addition of sulfite was further exemplified in the absence of pulp.

### Example 6

The stability of nitrogen-bound active halogen species in the presence of residual H₂O₂ was investigated. Free and total chlorine concentrations were measured by standard DPD methodology and the total H₂O₂ concentration by acidic sulfite titration. The concentration of MCDMH is the concentration of the total active halogen less the concentration of free active halogen. The concentration of H₂O₂ is the total oxidant concentration less the MCDMH concentration. Combination of 2.1 ppm (0.062 mM) H₂O₂ with 1 ppm (0.014 mM) NaOCl (as Cl₂) resulted in an immediate stoichiometric decrease in both materials, leaving a H₂O₂ residue of ˜1.6 ppm (0.048 mM) with no detectable free chlorine. The indicated reaction is shown in Equation 6.

NaOCl+H₂O₂→H₂O+NaCl+O₂ (6)

The inherent instability of active halogen in the presence of H₂O₂ is shown in Table 6.

TABLE 6

| | Analyzed Residual | | | | |
|---|---|---|---|---|---|
| | Free active | Total active | | Indicated Species | |
| Time | halogen DPD | halogen DPD | Total Oxidants¹⁾ | | NaOCl (as Cl₂) |
| [min] | (as Cl₂) [ppm] | (as Cl₂) [ppm] | (as H₂O₂) [ppm] | H₂O₂ [ppm] | [ppm] |
| 0 | — | — | — | 2.1 (theory) | 1.0 (theory) |
| 1 | 0.0 | 0.0 | 1.8 | 1.8 | 0.0 |
| 5 | — | — | 1.8 | 1.8 | 0.0 |
| 15 | — | — | 1.4 | 1.4 | 0.0 |

¹⁾Determined using HACH HYP-1 hydrogen peroxide test kit (Hach Co., Loveland, CO)

### Example 7

The effect of the addition of a molar equivalent of 5,5-dimethylhydantoin (DMH) to NaOCl solutions prior to combination with hydrogen peroxide was investigated. The results are shown in Table 7. The concentration of MCDMH is the concentration of the total active halogen less the concentration of free active halogen. The concentration of H₂O₂ is the total oxidant concentration less the MCDMH concentration.

TABLE 7

| | Analyzed Residual | | | | |
|---|---|---|---|---|---|
| | Free active | Total active | | Indicated Species | |
| Time | halogen DPD | halogen DPD | Total Oxidants¹⁾ | | DMH stabilized |
| [min] | (as Cl₂) [ppm] | (as Cl₂) [ppm] | (as H₂O₂) [ppm] | H₂O₂ [ppm] | NaOCl (as Cl₂) [ppm] |
| 0 | — | — | — | 2.1 | 1.0 |
| 1 | 0.0 | 0.9 | 2.6 | 2.1 | 0.9 |
| 5 | 0.0 | 1.0 | 2.4 | 1.9 | 1.0 |
| 15 | 0.0 | 0.9 | — | — | 0.9 |
| 60 | 0.0 | 0.9 | 2.8 | 2.3 | 0.9 |

¹⁾Determined using HACH HYP-1 hydrogen peroxide test kit (Hach Co., Loveland, CO)

It appears that the addition of DMH stabilizes both active chlorine and hydrogen peroxide upon combination. No significant decomposition was observed even after 1 h contact time.

1. A method for controlling microbial growth in an aqueous system containing sulfite and/or bisulfite residues, said method comprising the addition of a composition comprising at least one peroxy compound to said aqueous system at a pH greater than about 5.

2. The method of claim 1, wherein the peroxy compound is selected from the group consisting of hydrogen peroxide, alkali metal percarbonates, alkaline earth metal percarbonates, alkali metal perborates, alkaline earth metal perborates, alkali metal persulfates, alkaline earth metal persulfates, organic peroxy acids, and mixtures thereof.

3. The method of claim 2, wherein said composition further comprises a bleach activator.

4. The method of claim 3, wherein said bleach activator is tetraacetylethylendiamine.

5. The method of claim 2 where the peroxy compound is hydrogen peroxide.

6. The method of claim 1, wherein the pH is between about 6 and about 11.

7. The method of claim 6, wherein the pH is between about 7.5 and about 10.

8. The method of claim 1, wherein the pH is greater than about 9.

9. The method of claim 1, wherein the pH of the aqueous system is adjusted using a compound selected from the group consisting of alkali metal hydroxides, alkaline earth metal hydroxides, alkali metal bicarbonates, alkaline earth metal bicarbonates, alkali metal carbonates, alkaline earth metal carbonates, alkali metal metasilicates, or mixtures thereof.

10. The method of claim 1, wherein the concentrations of sulfite and/or bisulfite and peroxy compound immediately after addition are 1 to 300 ppm each.

11. A method for stabilizing an active halogen biocide in a peroxide-containing aqueous system, said method comprising the addition of an N-hydrogen compound to the active halogen biocide before combining it with said peroxide-containing aqueous system.

12. The method of claim 11, wherein the concentration of active halogen stabilized by N-hydrogen compound (as Cl₂) is 0.1 to 20 ppm.

13. The method of claim 11, wherein the N-hydrogen compound is selected from the group consisting of ammonia, ammonium salts, such as ammonium sulfate and ammonium bromide, nitrogen compounds containing no carbon-hydrogen bonds, such as urea, biuret, sulfamic acid, and isocyanuric acid, substituted N-hydrogen compounds such as methane-sulfonamide, p-toluenesulfonamide, 5,5-dialkylhydantoins, barbituric acid, 5-methyluracil, imidazoline, pyrrolidone, morpholine, acetanilide, acetamide, N-ethylacetamide, phthalimide, benzamide, succinimide, N-methylolurea, N-methylurea, acetylurea, methyl allophanate, methyl carbamate, phthalohydrazide, pyrrole, indole, formamide, N-methyl-formamide, dicyanodiamide, ethyl carbamate, 1,3-dimethylbiuret, methylphenylbiuret, 4,4-dimethyl-2-oxazolidinone, 6-methyluracil, 2-imidazolidinone, ethyleneurea, 2-pyrimidone, azetidin-2-one, 2-pyrrolidone, caprolactam, phenylsulfinimide, phenylsulfinimidylamide, diarylsulfinimides, dialkylsulfinimides, isothiazoline-1,1-dioxide, hydantoin, glycine, piperidine, piperazine, ethanolamine, glycinamide, creatine, glycoluril, and mixtures thereof.

14. The method of claim 13, wherein the N-hydrogen compound is 5,5-dimethylhydantoin.

15. The method of claim 13, wherein the N-hydrogen compound is urea, ammonia, or an ammonium salt.

16. The method of claim 11, wherein the peroxide is selected from the group consisting of hydrogen peroxide, alkali metal percarbonates, alkaline earth metal percarbonates, alkali metal perborates, alkaline earth metal perborates, alkali metal persulfates, alkaline earth metal persulfates, organic peroxy acids, and mixtures thereof.

17. The method of claim 16, wherein the peroxide is hydrogen peroxide.

18. The method of claim 11, wherein the aqueous system is selected from the group consisting of pulp and papermaking slurries, recycle pulp slurries, pulp thick stock, deinking pulp slurries, pulp or biomass bleaching slurries and liquids, textile bleaching solutions and clay slurries.

19. The method of claim 11, wherein the aqueous system is selected from the group consisting of waste water, papermaking liquors and waters, pool and spa waters, industrial cooling waters, waters exposed to reverse osmosis filters or ion exchange resins, and aqueous systems in oil field applications, including fractionation tanks and down hole applications.

20. The method of claim 11, wherein the aqueous system is selected from aqueous solutions for food and crop protection applications, including fruit and vegetable washes, meat and poultry processing, beverage processing, fish farming and aquaculture.

21. The method of claim 11, wherein the aqueous system containing peroxides has been obtained by the addition of a peroxy compound to an aqueous system containing sulfite and/or bisulfite residues at a pH greater than about 5.

22. A method for determining peroxide concentrations in aqueous systems in the presence of sulfite and/or bisulfite, said method comprising the steps of
(i) adding a defined excess of an N-hydrogen-stabilized active chlorine compound to immediately destroy the sulfite and/or bisulfite while leaving an amount of unreacted N-hydrogen stabilized active chlorine compound,
(ii) measuring the amount of unreacted N-hydrogen-stabilized active chlorine compound to determine the sulfite and/or bisulfite concentration, and
(iii) determining the peroxide concentration.

23. The method of claim 22, wherein the N-hydrogen stabilized active chlorine compound is 1-chloro-5,5-dimethylhydantoin.
